(19) United States
(12) Patent Application Publication
(10) Pub. No.: US 2023/0355380 A1
(43) Pub. Date: Nov. 9, 2023

(54) SYSTEMS, METHODS, AND DEVICES FOR EXPANDABLE SENSORS

(71) Applicant: SHIFAMED HOLDINGS, LLC, Campbell, CA (US)

(72) Inventors: Claudio ARGENTO, Felton, CA (US); Andrew BACKUS, Santa Cruz, CA (US)

(21) Appl. No.: 18/246,307

(22) PCT Filed: Sep. 22, 2021
(86) PCT No.: PCT/US2021/051475

§ 371 Date: Mar. 22, 2023

Related U.S. Application Data
(60) Provisional application No. 63/081,825, filed on Sep. 22, 2020.

(51) Int. Cl. A61F 2/24; A61B 5/00

(52) U.S. Cl. CPC A61F 2/2418; A61B 5/0031; A61B 5/6862; A61F 2250/0096; A61F 2250/0002

(57) ABSTRACT

Systems comprising expandable devices for making wireless measurements at a target tissue of a patient are disclosed herein. Configurations of expandable devices addressing potential negative consequences of mechanical loading of sensors and circuitry of the expandable devices during delivery and expansion at a target location are disclosed. Systems can comprise an external device configured to provide electrical power to the expandable device and to communicate data wirelessly with the expandable device.

### CROSS-REFERENCE TO RELATED APPLICATIONS

This application claims priority to U.S. Provisional Patent Application No. 63/081,825, filed on Sep. 22, 2020, titled “SYSTEMS, METHODS, AND DEVICES FOR EXPANDABLE SENSORS,” the entirety of which is incorporated by reference herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Expandable frame devices are useful in various medical capacities, including for applications such as stenting open biological structures and/or providing mechanical support during surgical procedures.

In general, it is advantageous to maximize the difference between at least one dimension of an expanded configuration and a collapsed configuration of an expandable frame device so that the expandable frame device can be delivered to a target location inside of a subject in as minimally invasive a manner as possible. To facilitate large differences in at least one dimension (of an expandable frame device when the device is in an expanded configuration versus when it is in a collapsed configuration), expandable frame devices generally comprise materials and geometries suited for bending.

It can be advantageous for expandable frame devices to measure, record, transmit, or receive data when used in various medical capacities. However, efforts to produce expandable frame devices having the capability to measure, record, transmit, or receive data have faced significant technical challenges. Materials useful for constructing a circuit comprising a sensor, a processor, or a transmitter are generally not well-suited for the physical deformation commonly associated with deployment, expansion, and/or collapse of expandable frame devices.

Accordingly, there exists a need for improved expandable frame devices capable of measuring, recording, transmitting, or receiving data that can endure physical forces associated with expansion and/or collapse of the expandable frame devices.

### SUMMARY

Implantable, expandable devices configured to withstand the loading and unloading of structural elements, particularly implantable devices comprising one or more sensors, can reduce the likelihood that the device would suffer failure during deployment or expansion. It would therefore be desirable to provide expandable devices comprising a sensor and configured to sustain mechanical loading and unloading of the frame structure of the expandable device. Not necessarily all such aspects or advantages are achieved by any particular embodiment. Thus, various embodiments may be carried out in a manner that achieves or optimizes one advantage or group of advantages taught herein without necessarily achieving other aspects or advantages as may also be taught or suggested herein.

The present disclosure generally relates to expandable devices and systems comprising sensors and more particularly relates to deployment of prosthetic heart valves comprising sensors at or near a diseased native valve in a patient.

An aspect of the present disclosure provides an expandable device. In some embodiments, the expandable device comprises a frame structure having an expanded configuration and an unexpanded configuration. In some embodiments, the expandable device comprises one or more sensors directly coupled to one or more struts of the frame structure. In some embodiments, the expandable device comprises one or more antennas coupled to the one or more sensors. In some embodiments, the expandable device comprises a frame structure having an expanded configuration and an unexpanded configuration; one or more sensors directly coupled to one or more struts of the frame structure; and one or more antennas coupled to the one or more sensors.

In some embodiments, the one or more antennas are coupled directly to the frame structure. In some embodiments, at least one surface of an antenna of the one or more antennas comprises a non-conducting coating. In some embodiments, an antenna of the one or more antennas comprises an induction coil.

In some embodiments an expandable device further comprises one or more on-board circuits. In some embodiments, an on-board circuit of the one or more on-board circuits is electrically coupled to the one or more sensors. In some embodiments, the one or more on-board circuits comprise a processor configured to convert data received from the one or more sensors into one or more transmission signals. In some embodiments, the one or more on-board circuits comprise a processor configured to operate the one or more sensors based on a signal received by the one or more antennas.

In some embodiments, the frame structure comprises a plurality of struts coupled at a plurality of strut joints. In some embodiments, each sensor of the one or more sensors is coupled to the frame structure between two strut joints. In some embodiments, each on-board circuit of the one or more on-board circuits is coupled to the frame structure between two strut joints. In some embodiments, the two strut joints are adjacent strut joints.

In some embodiments, the expandable device comprises a plurality of sensors. In some embodiments, a first sensor of the plurality of sensors is coupled to the frame structure closer to a first longitudinal end of the frame structure than to a second longitudinal end of the frame structure, and a second sensor of the plurality of sensors is coupled to the frame structure closer to the second longitudinal end of the frame structure than to the first longitudinal end of the frame structure.

Another aspect of the present disclosure provides a system for treating a system comprising an expandable device disclosed herein. In some embodiments, the system comprises an external device. In some embodiments, the external device is configured to receive a signal from the one or more antennas of the expandable device. In some embodiments, a system for treating a system comprising an expandable device disclosed herein; and an external device configured to receive a signal from the one or more antennas of the expandable device.

In some embodiments, the one or more antennas are coupled directly to the frame structure. In some embodiments at least one of the one or more antennas comprises an insulator material. In some embodiments, at least one of the one or more antennas are coupled to the frame structure at an angle transverse to the longitudinal axis of the frame structure. In some embodiments, an antenna of the one or more antennas comprises an induction coil.

In some embodiments, the external device comprises a transmission coil.

In some embodiments, the external device comprises a computer, the computer comprising a processor and a non-transitory memory comprising instructions that cause the external device to transmit a signal wirelessly to the expandable device when executed by the processor. In some embodiments, the instructions cause the external device to process a signal received from the expandable device when executed by the processor. In some embodiments, processing the signal received from the expandable device comprises converting a frequency shift keying signal to a digital signal.

In some embodiments, the transmission coil is incorporated into a handheld wand. In some embodiments the transmission coil is coupled to an examination bed. In some embodiments, the transmission coil is coupled to a pad.

In some embodiments, the frame structure expands along an expansion axis when transitioning from the unexpanded configuration to the expanded configuration wherein the wire antenna is oriented in a direction that is over 45 degrees from the axis of expansion.

Another aspect of the present disclosure provides a method of deploying an expandable device. In some embodiments, a method of deploying an expandable device comprises delivering an expandable device disclosed herein to a target region of a subject. In some embodiments, the expandable device comprises a frame structure, one or more sensors coupled directly to the frame structure, and one or more antennas coupled directly to the frame structure. In some embodiments, a method of deploying an expandable device comprises expanding the expandable device at the target region of a subject. In some embodiments, a method of deploying an expandable device comprises delivering an expandable device disclosed herein to a target region of a subject, the expandable device comprising a frame structure, one or more sensors coupled directly to the frame structure, and one or more antennas coupled directly to the frame structure; and expanding the expandable device at the target region of a subject.

Another aspect of the present disclosure provides a method of receiving an analog signal with an expandable device. In some embodiments, a method of receiving an analog signal with an expandable device comprises receiving an encoded analog signal using a wire of the expandable device. In some embodiments, a method of receiving an analog signal with an expandable device comprises converting the signal from an analog signal to a digital signal. In some embodiments, a method of receiving an analog signal with an expandable device comprises operating one or more sensors coupled directly to the expandable device based on the digital signal. In some embodiments, a method of receiving an analog signal with an expandable device comprises receiving an encoded analog signal using a wire of the expandable device; converting the signal from an analog signal to a digital signal; and operating one or more sensors coupled directly to the expandable device based on the digital signal.

In some embodiments, a method of receiving an analog signal comprises transmitting the analog signal to the expandable device using an external device. In some embodiments, a method of receiving an analog signal comprise encoding the analog signal prior to transmitting the analog signal to the expandable device. In some embodiments, the analog signal is transmitted wirelessly. In some embodiments, the analog signal is received wirelessly. In some embodiments, the analog signal is encoded using on-off keying. In some embodiments, the analog signal comprises instructions for one or more sensors of the expandable device. In some embodiments, the analog signal is used to power a sensor of the one or more sensors.

In some embodiments, the sensor of the one or more sensors is powered through transcutaneous energy transfer. In some embodiments, the analog signal is a radio frequency signal.

An aspect of the present disclosure provides a method of transmitting data from an expandable device. In some embodiments, the method comprises encoding sensor data from one or more sensors directly coupled to the expandable device. In some embodiments, the method comprises transmitting the encoded sensor data to an external device. In some embodiments, a method of transmitting data from an expandable device comprises encoding sensor data from one or more sensors directly coupled to the expandable device and transmitting the encoded sensor data to an external device. In some embodiments, a method of transmitting data from an expandable device comprises generating the sensor data by using the one or more sensors to measure one or more parameters of a target location of a subject.

In some embodiments, the sensor data is encoded using frequency shift keying.

In some embodiments, a method disclosed herein comprises receiving the encoded sensor data using a hand-held wand of the external device. In some embodiments, the hand-held wand of the external device comprises a receiving coil configured to receive the encoded sensor data. In some embodiments, the encoded sensor data is transmitted using a wire of the expandable device. In some embodiments, the wire is coupled to one or more struts of the expandable device. In some embodiments, the wire is an inductive coil. In some embodiments, the wire is an antenna.

In some embodiments, a method disclosed herein comprises converting the encoded sensor data from an analog signal to a digital signal. In some embodiments, the encoded sensor data is converted from an encoded analog signal to a digital signal using a processor of the external device. In some embodiments, the method disclosed herein comprises displaying the sensor data. In some embodiments, the sensor data is displayed at a visual display of the external device. In some embodiments, a method of transmitting data further comprises storing the sensor data using a non-transient memory of the external device. In some embodiments, the analog signal is a radio frequency signal.

These and other embodiments are described in further detail in the following description related to the appended drawing figures.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 shows a side view of a device comprising a frame structure and a plurality of sensors, in accordance with some embodiments.

FIG. 2A shows a schematic of a device comprising a frame structure in an unexpanded configuration, in accordance with some embodiments.

FIG. 2B shows a schematic of the device of FIG. 2A in an expanded configuration, in accordance with some embodiments.

FIG. 3A shows a schematic of a device comprising a frame structure, a sensor, and a plurality of receiver wires, in accordance with some embodiments.

FIG. 3B shows a schematic of a device comprising a frame structure, a plurality of sensors, and a receiver wire, in accordance with some embodiments.

FIG. 4 shows a circuit diagram of a sensor circuit, in accordance with some embodiments.

FIG. 5 shows a circuit diagram of a remote component, in accordance with some embodiments.

FIG. 6 shows a diagram of an expandable sensor system used in a subject, in accordance with some embodiments.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The present disclosure is described in relation to systems and devices useful in sensing biological parameters in a subject and methods of use thereof. However, one of skill in the art will appreciate that this is not intended to be limiting and the devices and methods disclosed herein may be used in other anatomical areas and in other surgical procedures.

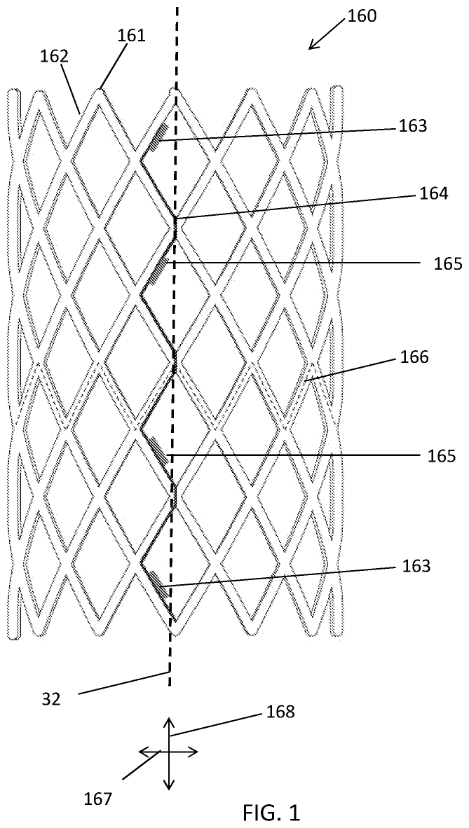

FIG. 1 shows an exemplary expandable device 160 comprising frame structure 161 and one or more sensors 163. In some embodiments, the sensor(s) 163 can be coupled to the frame structure 161 or a portion thereof, e.g., can be coupled to one or more struts 162 of frame structure 161. In some embodiments, the sensor(s) 163 can be coupled to a main wire 164 (e.g., an antenna or inductive coil) of expandable device 160. Further, the main wire 164 can be coupled to the frame structure 161 or a portion thereof. The main wire 164 can further be coupled to one or more an on-board circuit(s) 165, which can include a receiver wire, a microcontroller (e.g., a processor), and/or signal processing circuitry. The expandable device 160 can further include a receiver wire 166 (e.g., an antenna or inductive coil). The receiver wire 166 can be coupled (e.g., electrically coupled) to the main wire 164, the on-board circuit(s) 165, and/or the sensor(s) 163. The receiver wire 166 can include a material that is an electrical conductor. In some embodiments, the receiver wire 166 can have a shape of an open circle (e.g., an open ring) or a coil. For example, the receiver wire 166 can be a wire that encircles a circumference (e.g., a circumference measured along an outer surface) of the expandable device 160 one or more times (e.g., the receiver wire 166 can have one or more loops or coils). The receiver wire 166 can be an induction coil. Further, the receiver wire 166 can be a receiver wire and/or a transmission wire.

The sensor(s) 163 and associated circuitry (e.g., the main wire 164, the receiver wire 166, and/or the on-board circuit(s) 165) can allow the measurement of parameters in a region in which an expandable device 160 comprising the sensor is deployed. For example, the sensor(s) 163 can be used to measure one or more of: a strain (e.g., a deformation), a temperature, a fluid velocity, a force (e.g., a normal force or a shearing force), a pressure, a pH, or an oxygen concentration at or near the expandable device 160. The sensor(s) 163 can be capacitive sensors, resistive sensors, and/or piezoelectric sensors. In some embodiments, the expandable device 160 can include a plurality of the same type of sensor 163. For example, the expandable device 160 can include a plurality of flow rate sensors located at a plurality of locations along a longitudinal axis of the expandable device 160, e.g., to determine a flow rate through a portion of a subject's body. In some embodiments, the expandable device 160 can include two or more different types of sensors 163.

The sensor(s) 163 can be configured to receive a signal from a wire (e.g., the main wire 164 or receiver wire 166) or from the on-board circuit(s) 165 of expandable device 160. In some embodiments, the sensor(s) 163 can be configured to make a measurement after receiving a signal from the on-board circuit(s) 165 or from an external device. In some embodiments, the sensor(s) 163 can be configured to make a measurement when a measured parameter (e.g., a biological parameter measured by sensor(s) 163) reaches and/or exceeds a threshold value.

The sensor(s) 163 can be configured to transmit data (e.g., data from a measurement) to the on-board circuit(s) 165. For example, the sensor(s) 163 can be configured to send measurement data (e.g., through a wire of expandable device 160) to the on-board circuit(s) 165 for transmission to an external device.

In some embodiments, the expandable device 160 includes only one sensor 163. In other embodiments, the expandable device 160 includes a plurality of sensors 163. For example, the expandable device 160 can include 2, 3, 4, 5, 6, 7, 8, 9, 10, from 10 to 20, from 20 to 30, from 30 to 40, from 40 to 50, or more than 50 sensors 163. Embodiments of expandable devices 160 including only one sensor 163 can be useful in cases where measurements being made do not require multiple sensors. Embodiments of expandable devices 160 including a plurality of sensors 163 can be useful in cases where complex measurements are to be made. For example, the inclusion of a plurality of sensors 163 can be advantageous for making differential measurements and/or for making multiple types of measurements (e.g., strain, temperature, or chemical analysis).

The sensor(s) 163 and/or circuitry associated with sensor 163 (e.g., the main wire 164, the receiver wire 166, and/or the on-board circuit(s) 165) can be disposed on the frame structure 161 so as to reduce any negative effects on the sensor and/or circuitry's functionality or durability during transitioning frame structure 161 between the expanded configuration and unexpanded configuration.

Further, one or more of the sensor(s) 163, wire 164, on-board circuit(s) 165, or receiver wire 166 can be coupled to and/or oriented with respect a portion or axis of frame structure 161 to improve the function (e.g., sensing, change in configuration, and/or durability) or physical properties of expandable device 160 or a portion thereof. For example, to decrease fatigue of brittle or less ductile components of expandable device 160 (e.g., of the sensor(s) 163, wire 164, on-board circuit(s) 165, or receiver wire 166) that can result from a change in one or more dimensions of expandable device 160 (e.g., as a result of transitioning between the unexpanded and expanded configurations), the one or more portions of the expandable device 160 can be coupled to the frame structure 161 in an orientation that is not along or parallel to an axis of expansion (e.g., axes of expansion 167, 168) of expandable device 160. For example, the one or more portions of the expandable device 160 (e.g., sensor(s) 163, main wire 164, on-board circuit(s) 165, and/or receiver wires 166) can be oriented at an angle that is from 30 to 45 degrees, from 45 degrees to 60 degrees, or from 60 degrees to 90 degrees from an axis of expansion of expandable device 160. In other embodiments, one or more of the sensor(s) 163, main wire 164, on-board circuit(s) 165, or receiver wire 166 can be coupled to the frame structure 161 in an orientation that is in-line with (e.g., along or parallel to) an axis of expansion of expandable device 160. For example, one or more one or more of the sensor(s) 163, wires 164, on-board circuit(s) 165, or receiver wire 166 can be coupled to the frame structure 161 in an orientation within or along a plane perpendicular to (e.g., transverse to) the longitudinal axis of expandable device 160 or a portion thereof (e.g., the frame structure 161).

As described herein, the frame structure 161 can have an expanded configuration and an unexpanded (e.g., collapsed) configuration. In some embodiments, the frame structure 161 can have a different length, circumference, radius, width, height, or cross-sectional area in one or more directions when in a first configuration (e.g., the unexpanded configuration) than when it is in a second configuration (e.g., the expanded configuration). For example, a frame structure 161 shaped like a tube or cylinder can have a larger radius when it is in an expanded configuration than it has when it is in an unexpanded configuration (e.g., for at least a portion of the longitudinal length of the frame structure). In some embodiments, a tubular frame structure 161 can decrease in longitudinal length (e.g., as measured along a longitudinal axis 32 of frame structure 161) and increase in radius when changing from an unexpanded configuration to an expanded configuration. In some embodiments, the frame structure 161 can have one or more axes of expansion.

The frame structure 161 can be configured to be expanded at or within a target region (e.g., a target location) of a subject. For example, the frame structure 161 can be expanded at the target region to assume an expanded configuration. Further, the frame structure 161 can be delivered to the target region of a subject in an unexpanded configuration (e.g., within a catheter or sheath). In some embodiments, the frame structure 161 can be tubular (e.g., cylindrical) in shape. The frame structure 161 can include a plurality of interconnected struts 162. In some embodiments, the plurality of struts 162 of the frame structure 161 can make up an expandable web or mesh. In some embodiments, the shape (e.g., length, curvature, and/or cross-sectional shape) of the one or more struts 162 of frame structure 161 and/or the angle at which a strut 162 of frame structure 161 is coupled to one or more additional struts 162 of frame structure 161 can affect the strength of frame structure 161, e.g., in resisting external compressive forces or maintaining its shape under external compressive forces. For example, the plurality of struts 162 of frame structure 161 can include a plurality of expandable cells forming a web or mesh. In some embodiments, the one or more expandable cells of a frame structure 161 can aid in the ability of the frame structure 161 to assume the unexpanded configuration during delivery while maintaining the ability of the frame structure 161 to maintain its shape under a physical force after being deployed into the expanded configuration.

The frame structure 161 can be made of one or more materials. In some embodiments, the frame structure 161 or a portion thereof can include a plurality of different materials. Materials that can be make up the frame structure 161 or a portion thereof include: metals, metal alloys, polymers, co-polymers, and ceramics. The frame structure 161 or a portion thereof can include a rigid material, a semi-rigid material, a resilient material, or a flexible material. For example, a strut joint of the frame structure 161 can include a material capable of deforming under physical stress and returning to its original shape (or a shape substantially close to its original shape) when the physical stress is removed. In some embodiments, a strut joint of the frame structure 161 can include a material that allows the strut joint to act as a living hinge. In some embodiments, the frame structure 161 or a portion thereof can comprise an insulator. For example, the frame structure 161 can include a plurality of metal struts with an insulator material on at least one aspect of one or more of the metal struts (e.g., to insulate a conductor coupled to the frame structure, such as an antenna or coil, from a metal comprising the frame structure).

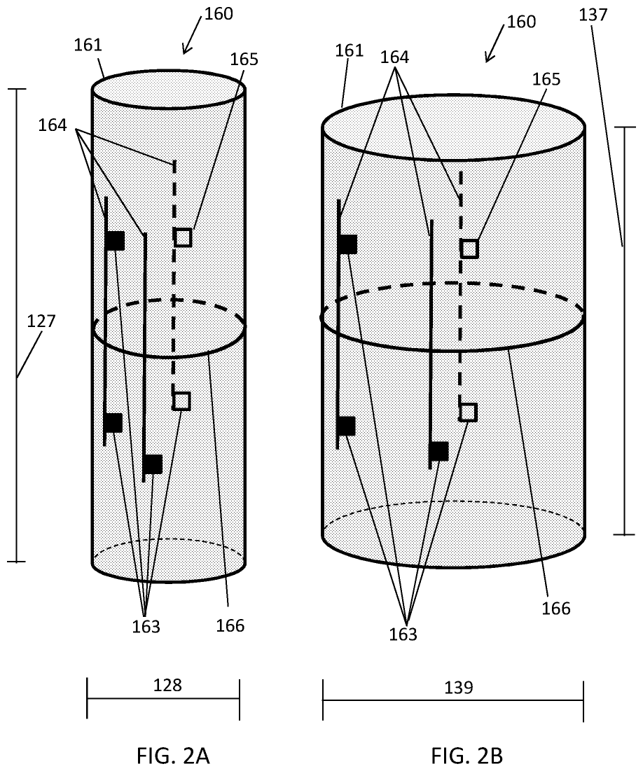

FIG. 2A shows a representative diagram of the frame structure 161 of the expandable device 160 in the unexpanded configuration. FIG. 2B shows a representative diagram of the frame structure 161 of the expandable device 160 in an expanded configuration. The frame structure 161 can have a cross-sectional diameter 128 in the unexpanded configuration. The frame structure 161 can have a cross-sectional diameter 139 in the expanded configuration. The cross-sectional diameter 128 of the frame structure 161 in the unexpanded configuration can be different from the cross-sectional diameter 139 of the frame structure 161 in the expanded configuration. For example, the cross-sectional diameter 128 can be smaller than the cross-sectional diameter 139. In some embodiments, the diameter 128 of the expandable device 160 or portion thereof (e.g., the frame structure 161) in the unexpanded configuration can be from 0.01 mm to 20 mm, 0.01 mm to 15 mm, 0.01 mm to 10 mm, from 0.01 mm to 9 mm, from 0.01 mm to 8 mm, from 0.01 mm to 7 mm, from 0.01 mm to 6 mm, from 0.01 mm to 5 mm, from 0.01 mm to 4 mm, from 0.01 mm to 3 mm, from 0.01 mm to 2 mm, from 0.01 mm to 1 mm, from 1 mm to 15 mm, from 2 mm to 14 mm, from 3 mm to 13 mm, from 4 mm to 12 mm, from 5 mm to 10 mm, from 6 mm to 10 mm, from 7 mm to 10 mm, from 8 mm to 10 mm, from 9 mm to 10 mm, from 10 mm to 15 mm, no more than 20 mm, no more than 15 mm, no more than 10 mm, no more than 9 mm, no more than 8 mm, no more than 7 mm, no more than 6 mm, or no more than 5 mm. In some embodiments, the diameter 139 of the expandable device 160 or a portion thereof (e.g., the frame structure 161) in the expanded configuration can be from 10 mm to 50 mm, from 20 mm to 40 mm, from 25 mm to 35 mm, from 27 mm to 33 mm, no more than 50 mm, no more than 40 mm, no more than 35 mm, no more than 33 mm, no more than 30 mm, no more than 25 mm, no more than 20 mm, or no more than 15 mm when frame structure 161 is in an expanded configuration.

In some embodiments, the diameter 139 in the expanded configuration refers to a largest cross-sectional width of the expandable device 160 or a portion thereof, e.g., as measured in a plane perpendicular to a longitudinal axis of expandable device 160 at a longitudinal location. In some embodiments, the expandable device 160 has a polygonal cross-section. In some embodiments, the diameter 139 can refer to the largest distance from a first side of a polygonal cross-section of expandable device 160 to a second side of the polygonal cross-section of the expandable device 160.

The frame structure 161 can have a length 127 in the unexpanded configuration and a length 137 in the expanded configuration. In some embodiments, the frame structure length 127 in the unexpanded configuration is different from the frame structure height 137 in the expanded configuration. For example, the frame structure length 127 in the unexpanded configuration can be greater than frame structure length 137 in the expanded configuration. In some embodiments, the frame structure length 127 in the unexpanded configuration can be the same as the frame structure length 137 in the expanded configuration.

In some embodiments, the expandable device 160 can be delivered to the target tissue location in the unexpanded configuration. The expandable device 160 may be balloon-expandable, self-expanding, or otherwise expansible as will be understood to one of ordinary skill in the art based on the teachings herein. In some embodiments, the expandable device 160 can be reconfigured from the unexpanded configuration to the expanded configuration at the target tissue location (e.g., by expanding with a malecot or balloon).

Minimizing the diameter 128 of the expandable device 160 when the expandable device 160 is in the unexpanded configuration can be advantageous for delivery of the expandable device 160. For example, an expandable device 160 with a smaller diameter can fit inside of a delivery device with a smaller diameter, allowing for less invasive delivery and for improved maneuvering capability inside of a subject's body. Further, reducing the diameter 128 of the expandable device 160 in the unexpanded configuration (e.g., for use in treatment or replacement of a mitral valve, a tricuspid valve, an aortic valve, or a pulmonic valve) can allow for easier delivery of the expandable device 160 to a target region of a subject, faster recovery of a subject receiving the expandable device 160, and/or improved clinical outcomes for a subject receiving the expandable device 160 (e.g., improved subject survival, improved ejection fraction, improved cardiac output, decreased valvular regurgitation, and/or decreased edema). In some embodiments, reducing the diameter 128 of the expandable device 160 in the unexpanded configuration can make transseptal access and delivery possible in addition to transapical access.

In some embodiments, the expandable device 160 or a portion thereof can be sized or shaped to be positioned at a certain location or target region. For example, the frame structure 161 can be sized to be positioned in a heart valve, such as the mitral valve (e.g., by designing a dimension of frame structure 161 to fit a valve, such as the mitral valve, when in an expanded configuration).

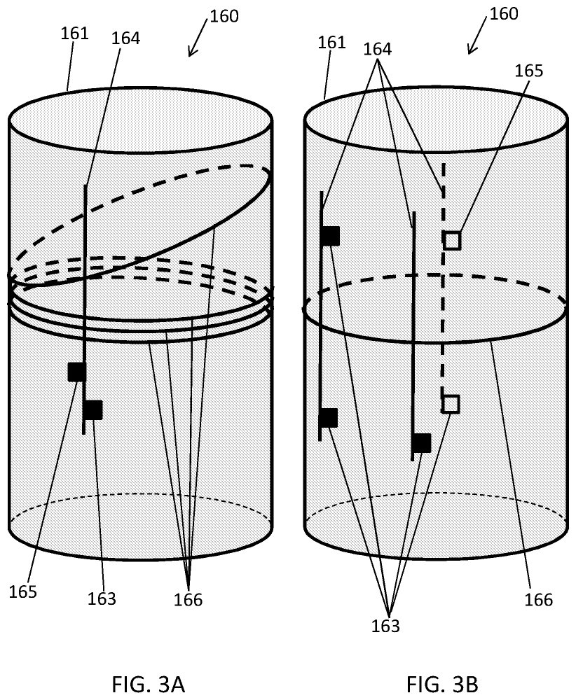

In some embodiments, the expandable device 160 can include a plurality of receiver wires 166 and/or main wires 164. In some embodiments, the inclusion of a plurality of wires (e.g., main 164 or receiver wires 166) can provide a wider range of angles at which a magnetic field or transmitted signal can be received or transmitted by the wires 164, 166 of expandable device 160. In some embodiments, an expandable device 160 including a wide range of angles at which a magnetic field, an electrical field, or a transmitted signal can be received or transmitted can, in turn, be more efficient at transmitting or receiving a signal and/or at undergoing inductive charging (e.g., transcutaneous energy transfer (TET) or resonance charging). FIG. 3A shows an embodiment of expandable device 160 including a plurality of receiver wires 166. As shown, at least one receiver wire 166 can be oriented differently than other expandable receiver wires 166 of the expandable device 160. FIG. 3B shows an embodiment of expandable device 160 including a plurality of sensors 163 and plurality of main wires 164. In some embodiments, inclusion of a plurality of sensors 163 and main wires 164 can allow data from sensors 163 disposed at different locations on expandable device 160 to be transmitted to an external receiver. In some embodiments, the inclusion of a plurality of main wires 164 reduces or eliminates the need to orient one or more main wires 164 in a direction in-line with or substantially in-line with an axis of expansion. Thus, in some embodiments, the inclusion of a plurality of main wires 164 can reduce the number of times and/or the degree to which a main wire 164 of expandable device 160 is mechanically loaded and/or unloaded (e.g., when expandable device 160 is expanded or deployed in a target region of a subject).

The connections between one or more of the main wires 164, on-board circuits 165, sensors 163, and/or receiver wires 166 of the expandable device 160 can be configured to optimize the distribution and/or orientation of the one or more sensors 163 and/or the one or more receiver wires 166, for example, as shown in FIG. 3A and FIG. 3B.

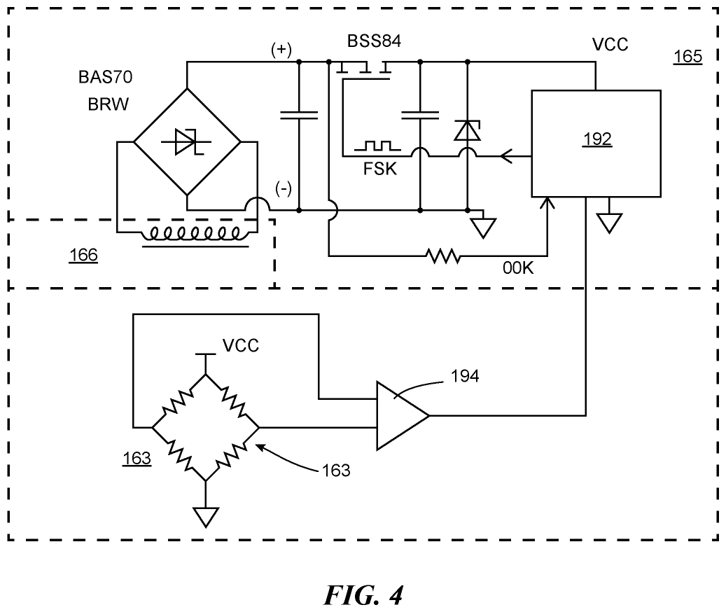

FIG. 4 shows a circuit diagram of a representative example of an expandable device 160, as disclosed herein. The sensor 163 of the expandable device 160 can be a resistive sensor. For example, the sensor 163 can be a pressure sensor (e.g., a pressure gauge). The sensor 163 can be electrically coupled to the on-board circuit 165. In some embodiments, the sensor 163 can pass data (e.g., in the form of electrical signal(s)) to the on-board circuit 165 via main wire 164.

The on-board circuits 165 described herein can be electrically coupled to a sensor 163 via the one or more main wires 164. The on-board circuit 165 can be electrically coupled to a sensor 163 via the one or more receiver wires 166, or a portion thereof. In some embodiments, the on-board circuit 165 can be electrically coupled to one or more sensors 163 via the one or more main wires 164 and the one or more receiver wires 166. For example, an on-board circuit 165 coupled to a first wire 164 can be electrically coupled to at least one sensor 163 via one or more receiver wires 166 (e.g., wherein the at least one sensor 163 is coupled to one or more second wires 164 and the first and second wires 164 are coupled to one another via one or more receiver wires 166). An on-board circuit 165 can, in some embodiments, be coupled to only one sensor 163. In other embodiments, the on-board circuit 165 can be electrically coupled to a plurality of sensors 163. In some embodiments, each on-board circuit 165 of one or more on-board circuits can be individually electrically coupled to a different sensor 163 of a plurality of sensors, for example, via one or more wires 164 and/or one or more receiver wires 166. In some embodiments, an on-board circuit 165 can be electrically coupled to a plurality of receiver wires 164 (e.g., via one or more wires 164).

The main wires 164 described herein can be made of a conductive metal, such as copper, platinum, gold, silver, aluminum, iron, or an alloy thereof. In some embodiments, the main wire 164 can function as an antenna wire. As described herein, it can be advantageous to limit the frequency and/or degree to which the main wire 164 is loaded and/or unloaded (e.g., when expandable device is delivered to a target region of a subject or when expandable device is expanded). The main wire 164 can be electrically isolated from at least a portion of the frame structure 161. For example, the main wire 164 can be partially or completely covered in an insulating material, such as a non-conductive plastic. In some embodiments the main wire 164 can be disposed on an outer surface of expandable device 160. For example, the main wire 164 can be disposed on an aspect of a component of the expandable device 160 that is radially more distant from a longitudinal axis 32 than the component itself. In some embodiments, the disposition of the main wire 164 on the outer surface of expandable device 160 can improve wireless signal transmission and/or wireless signal reception, especially when the frame structure of expandable device 160 comprises a conductor. In some embodiments, an insulating material can be disposed between the main wire 164 and the portion of frame structure 161 to which main wire 164 is coupled. In some embodiments (e.g., wherein the main wire 164 is an antenna wire), electrically isolating the main wire 164 from the frame structure 161 can improve signal transmission. In some embodiments, the main wire 164 can couple one or more sensors 163 to one or more on-board circuits 165. In some embodiments, an electrical signal from a sensor 163 can be passed through a bridge circuit (e.g., a Wheatstone bridge circuit or a Kelvin Double bridge circuit) and/or an operational amplifier before being passed to the on-board circuit 165.

In some embodiments, the expandable device 160 can include a plurality of on-board circuits 165. A first on-board circuit of a plurality of on-board circuits 165 of expandable device 160 can be electrically coupled to (e.g., in electrical communication with) a second on-board circuit of the plurality of on-board circuits 165. In some embodiments, distributing portions of signal processing circuitry comprising the expandable device 160 into a plurality of on-board circuits 165 can decrease the physical size of a component of expandable device 160 for housing the signal processing circuitry of expandable device 160. For example, distributing signal processing circuitry (and/or one or more processors associated with signal processing) can allow for two on-board circuits to be located on expandable device 160 instead of one on-board circuit that is larger in physical size than either of the two on-board circuits comprising equivalent signal processing circuitry.

The use of a plurality of relatively small on-board circuits 165 can decrease the likelihood that the circuitry of the expandable device 160 is negatively affected by a change in shape of expandable device 160 (e.g., when the expandable device 160 is delivered to a target tissue or when the expandable device 160 undergoes a transition from an unexpanded configuration to an expanded configuration). In some embodiments, portions of the signal processing circuitry of the expandable device 160 can be divided across a plurality of on-board circuits 165 that are sized such that an on-board circuit 165 of the plurality of on-board circuits can be coupled to the frame structure 161 between points of the frame structure 161 that are expected to experience loading or deformation while the expandable device 160 is delivered to a target tissue or expanded at a target region of a target tissue. For example, an on-board circuit 165 can have a length less than the distance between two points on a frame structure that are expected to experience loading (e.g., strut intersection points on a mesh frame structure) and/or a width equal to or less than that of a strut 163 of the frame structure 161 to which it is attached.

Referring back to FIG. 4, the on-board circuit(s) 165 can include one or more components for processing a signal. In some embodiments, the on-board circuit(s) 165 can include a microcontroller 192 (MCU), such as an ATtiny® 8-bit processor. In some embodiments, the on-board circuit(s) 165 can include a bridge circuit (e.g., a Wheatstone bridge circuit or a Kelvin Double bridge circuit) and/or an operational amplifier 194 (e.g., a zero drift, low offset, low power operational amplifier, such as OPA330) configured to process a signal from a sensor 163 before the signal is passed to another component of the on-board circuit 165, such as a microcontroller (e.g., a processor).

In some embodiments, an on-board circuit 165 of the expandable device 160 can include a non-transient memory for storing data measured by a sensor 163. For example, data collected using one or more sensors 163 can be stored by the on-board circuit 165 for subsequent transmission (e.g., wireless transmission) to an external receiver. Aggregation of a plurality of sensor signals (e.g., comprising a plurality of measurements made by one or more sensors 163) can be advantageous in that it can allow for batch transmission of sensor data to an external receiver (e.g., rather than requiring that a receiver be active and in range at all times that a sensor makes a measurement). An on-board circuit 165 that comprises a memory can also be advantageous for collection (e.g., aggregation and/or storage) and transmission of multiple data points over a range of time. In some embodiments, an on-board circuit 165 can store less than 1 minute, 1 minute to 5 minutes, 5 minutes to 10 minutes, 10 minutes to 15 minutes, 15 minutes to 30 minutes, 30 minutes to 1 hour, 1 hour to 2 hours, 2 hours to 6 hours, 6 hours to 12 hours, 12 hours to 24 hours, or more than 24 hours of data. Batch transmission can allow for aggregation of data received from a plurality of different sensors and transmission in a single signal.

The on-board circuit(s) 165 can also include one or more components for processing a signal a wire of expandable device 160 (e.g., receiver wire 166, which can comprise a ferrite antenna). For example, a rectifier bridge (e.g., a Zener diode rectifier bridge) can be used to normalize a received sinusoidal signal. In some embodiments, the rectifier bridge can include a smoothing capacitor.

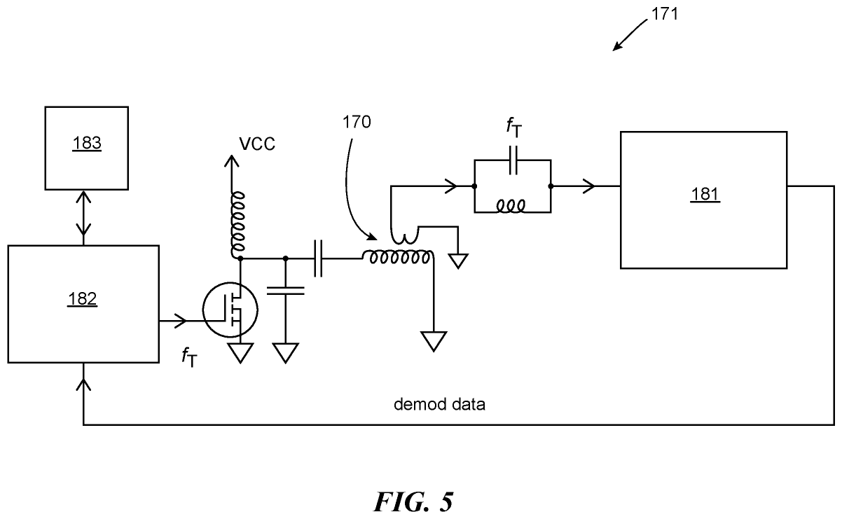

The expandable device 160 or a portion thereof (e.g., on-board circuit 165) can include a circuit configured to convert the signal from an analog signal to a digital signal, such as an on-off keying circuit (e.g., “OOK” in FIG. 4). An on-off keying circuit can be useful for processing an analog signal received from an outside source (e.g., an external device as described with respect to FIG. 5) into a binary signal useful to a microcontroller of the on-board circuit 165. For example, data, such as commands to records or transmit sensor data, can be received by a wire (e.g., a receiver antenna coil) of the expandable device 160.

The expandable device 160 or a portion thereof (e.g., on-board circuit 165) can include a frequency shift keying circuit. A frequency shift keying circuit (e.g., “FSK” in FIG. 4) of the expandable device 160 can be configured to have modulation frequencies determining the binary switch. A frequency shift keying circuit can be used to send data (e.g., measured sensor data) to an external device or portion thereof (e.g., external wire 170) via frequency-modulated transmitted signals. A frequency shift keying circuit can be configured to have a modulation frequency of from 1 kHz to 10 kHz, from 10 kHz to 20 kHz, from 20 kHz to 30 kHz, from 30 kHz to 40 kHz, or from 40 kHz to 50 kHz. For example, an expandable device 160 comprising a frequency shift keying circuit can be configured to respond to a 67 kHz signal received by transmitting signals of 67 kHz+/−10 kHz and 67 kHz+/−20 kHz for “on” and “off” signals.

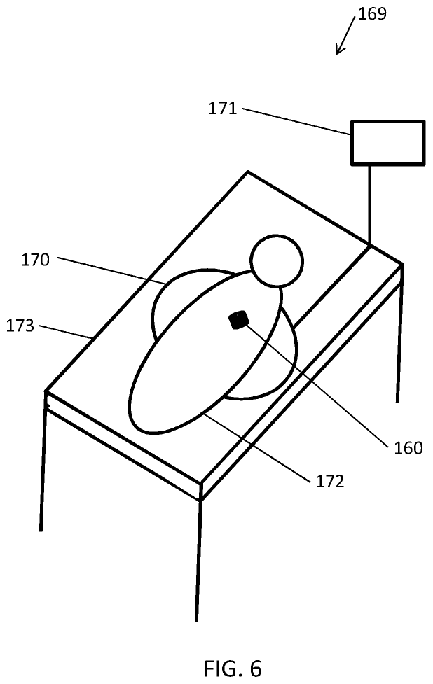

FIG. 6 shows a representative example of expandable sensor system 169 comprising an expandable device 160 deployed (e.g., implanted) at a target tissue of a subject 172. Subject 172 is shown lying on a bed or examination table, wherein an external wire 170 is located in close proximity to the patient (e.g., underneath the examination table, within the examination table, on a surface of the examination table, or within or on a material on top of the examination table, such as a pad). In some embodiments, the expandable sensor system 169 can be configured to detect one or more parameters in a target tissue of a subject 172. In some embodiments, the expandable sensor system 169 can include an external device 171 that is configured to wirelessly send data to and/or wirelessly receive data from the expandable device 160. In some embodiments, the external device 171 can be configured to provide electrical power to expandable device 160 (e.g., via an induction coil 170).

FIG. 5 shows a representative example of a circuit of an external portion of the expandable sensor system 169 including the external device 171 and external wire 170. In some embodiments, the external wire 170 can be a transmission coil. It will be appreciated by persons of skill in the art that external wire 170 can be used as a transmission coil and/or a receiver coil.

As shown in FIG. 5, the external device 171 of expandable sensor system 169 can include a receiver/demodulator module 181, which can include a processor (e.g., a DSP based receiver/demodulator, such as a ADAU1701 processor). The receiver/demodulator module 181 can receive a signal from the external wire 170, for example, after the external wire 170 has received the signal from a wire of expandable device 160. The receiver/demodulator module 181 can perform one or more signal processing functions on the received signal, such as converting a frequency shift keying signal to a digital signal. In many embodiments, data can be passed from the receiver/demodulator module 181 to a microcontroller of the external device 171. The external device 171 can further include a microcontroller (MCU) 182 and/or a universal asynchronous receiver/transmitter (UART) 183. A microcontroller 182 and/or UART 183 can be used to generate a waveform signal for transmission to the expandable device 160 of expandable sensor system 169. In some embodiments, a signal generated by external device 171 can be encoded for on-off keying by supplying power to and removing power from the transmission coil 170 of external device 171. In some embodiments, the external device 171 can include an amplifier. In some embodiments, the amplifier (e.g., a class E amplifier) of external device 171 can be coupled to the external wire 170. In some embodiments, the amplifier of external device 171 can be used to drive a signal through the external wire 170 (e.g., for transmission to an expandable device 160).

In some embodiments, power can be provided to the expandable device 160 via the external wire 170. For example, power can be induced in the receiver wire 166 by the external wire 170. In some embodiments, from 1 mW to 2 mW, from 2 mW to 3 mW, from 1 mW to 3 mW, from 0.5 mW to 4 mW, from 0.1 mW to 5 mW, or more than 5 mW can be provided to the receiver wire 166 of the expandable device 160 by the external device 171 (e.g., to power one or more components of expandable device 160, such as a microcontroller and/or a sensor). For example, 2 mW can be provided to the expandable device 160 wherein the expandable device 160 includes a microcontroller requiring 1 mW of power and a pressure-gauge sensor requiring 1 mW of power.

In one representative example, more than 2 W can be used to power an external device 171 having a 10 cm transmission coil 170 (e.g., within an external wand device) located 10 cm away from a 2×5 mm ferrite receiver wire 166 of the expandable device 160, e.g., to provide power and/or a signal to the receiver wire 166 of the expandable device 160.

In another representative example, more than 20 W can be used to power an external device 171 having a 50 cm transmission coil 170 (e.g., within a table or bed on which a subject is positioned or within a pad on which or under which the subject is positioned) located 50 cm from a 2×10 mm ferrite receiver wire 166 of an expandable device 160, e.g., to provide power and/or a signal to the receiver wire 166 of expandable device 160.

As described herein, the expandable device 160 can include a frame structure 161. The frame structure 161 can provide mechanical support to one or more components of the expandable device 160 (e.g., to one or more sensors 163, wires 164. 165, or on-board circuits 165 of the expandable device 160). The frame structure 161 can also provide mechanical support to one or more biological tissues of a subject, e.g., by exerting a force against the one or more biological tissues. For example, the frame structure 161 can provide mechanical support to a wall of a blood vessel or to a portion of the heart, such as a heart valve. In a representative example, the expandable device 160 comprises a frame structure 161 configured to exert a force radially outward against a heart valve when deployed in the orifice of the native heart valve.

The frame structure 161 can have an expanded configuration and an unexpanded configuration. In some embodiments, the expanded configuration of the frame structure 161 can be the same as the expanded configuration of the expandable device 160. In some embodiments, the unexpanded configuration of frame structure 161 is the same as the unexpanded configuration of the expandable device 160. In some embodiments, one or more dimension of the frame structure 161 is smaller when the frame structure 161 is in an unexpanded configuration than when frame structure 161 is in an expanded configuration. For example, a tube-shaped (e.g., tubular) frame structure 161 can have a smaller diameter when in an unexpanded configuration compared to when frame structure 161 is in an expanded configuration. The frame structure 161 can have a tubular or cylindrical shape comprising a longitudinal axis 32. The frame structure 161 can have a first longitudinal end and a second longitudinal end opposite the first longitudinal end.

The frame structure 161 can include a plurality of struts 162. In some embodiments, the strut 162 can be rigid (e.g., stiff). In some embodiments, the strut 162 can be semi-rigid (e.g., resilient or flexible). One or more struts 162 can be oriented and/or connected to one or more other structure to provide structural strength to frame structure 161. For example, one or more strut 162 of the frame structure 161 can be oriented parallel to a longitudinal axis 32 to provide structural strength to the frame structure (e.g., in response to compressive force in a longitudinal or substantially longitudinal direction relative to the longitudinal axis 32 of the frame structure).

One or more struts 162 of frame structure 161 can be oriented in a circumferential or substantially circumferential direction relative to the frame structure 161 (e.g., residing in a plane that is perpendicular or substantially perpendicular to a longitudinal axis 32). One or more struts 162 of the frame structure 161 can be oriented at an angle (e.g., an oblique, parallel, or perpendicular angle) relative to a cross-sectional plane that is perpendicular to longitudinal axis 32.

In some embodiments, the frame structure 161 can be a stent or an expandable heart valve. The frame structure 161 can be used to anchor the expandable device 160 in position at a target location of a subject (e.g., in the orifice of a heart valve, such as a mitral valve or tricuspid valve). At least a portion of the frame structure 161 can be a rigid (e.g., stiff) or semi-rigid (e.g., resilient or flexible) structure. In various embodiments, a rigid portion of the frame structure 161 or portion thereof can be from 0.01 mm to 50 mm in longitudinal length, from 1 mm to 45 mm in longitudinal length, from 10 mm to 40 mm in longitudinal length, from 20 mm to 30 mm in longitudinal length, from 30 mm to 40 mm in longitudinal length, from 25 mm to 35 mm in longitudinal length, from 27.5 mm to 32.5 mm in longitudinal length, from 10 mm to 20 mm in longitudinal length, from 0.01 mm to 10 mm in longitudinal length, from 0.01 mm to 9 mm in longitudinal length, from 0.01 mm to 8 mm in longitudinal length, from 0.01 mm to 7 mm in longitudinal length, from 0.01 mm to 6 mm in longitudinal length, from 0.01 mm to 5 mm in longitudinal length, from 0.01 mm to 4 mm in longitudinal length, from 0.01 mm to 3 mm in longitudinal length, from 0.01 mm to 2 mm in longitudinal length, or from 0.01 mm to 1 mm in longitudinal length. In some embodiments, a rigid portion of the frame structure 161 can be no more than 10 mm in longitudinal length, no more than 9 mm in longitudinal length, no more than 8 mm in longitudinal length, no more than 7 mm in longitudinal length, no more than 6 mm in longitudinal length, no more than 5 mm in longitudinal length, no more than 4 mm in longitudinal length, no more than 3 mm in longitudinal length, no more than 2 mm in longitudinal length, or no more than 1 mm in longitudinal length. A rigid portion of the frame structure 161 can include one or more struts, one or more arches, one or more commissural posts, one or more leaflet hoops (e.g., hoop structures), one or more flange struts, one or more flange bends, one or more coil grabbers, and/or one or more anchors. In some embodiments, a second portion of valve prosthesis device can be configured to affix to the native valve, e.g., with the aid of an outer anchor.

The frame structure 161 of the expandable device 160 can include one or more specialized rigid, semi-rigid, or flexible members. For example, the frame structure 161 can include a commissural post or a member to which a valve leaflet is coupled (e.g., a leaflet hoop).

In some embodiments, the angle of a strut 162 relative to a cross-sectional plane that is perpendicular to longitudinal axis 32 can depend on the configuration of the frame structure 161 and/or the expandable device 160. For example, one or more struts 162 of the frame structure 161 may be more perpendicular to a cross-sectional plane (e.g., that is perpendicular to longitudinal axis 32) when the frame structure 161 is in the unexpanded configuration than when the frame structure 161 is in the expanded position. In some embodiments, the angle between a strut 162 of the frame structure 161 and a cross-sectional plane perpendicular to the longitudinal axis 32 can be from 45 degrees to 55 degrees, from 40 degrees to 60 degrees, from 35 to 65 degrees, from 30 degrees to 70 degrees, from 25 degrees to 75 degrees, from 20 degrees to 80 degrees, from 15 degrees to 85 degrees or from 0 degrees to 90 degrees when frame structure 161 is in an expanded configuration. In some embodiments, the angle of one or more struts 162 of the frame structure 161 relative to a cross-sectional plane perpendicular to a longitudinal axis 32 can be the same or approximately the same when the frame structure 161 is in the expanded configuration versus when the frame structure 161 is in the unexpanded configuration.

A first strut (e.g., strut 162, commissural post, or leaflet hoop) of the frame structure 161 can be connected to a second strut (e.g., strut 162, commissural post, or leaflet hoop) of the frame structure 161 (e.g., at a strut joint). The frame structure 161 can include a plurality of struts 162 connected at a plurality of strut joints (e.g., forming a lattice structure, which can comprise a portion of frame structure 161). In some embodiments, one or more strut 162 of the frame structure 161 can exert a force against another structure. A first strut 162 can be coupled rigidly to a second strut 162. For example, a strut joint can include a weld or fastener. In some embodiments, a rigid strut joint does not allow one or more strut 162 connected to the joint to move freely at the joint. In some embodiments, a rigid strut joint can be continuous with one or more struts 162 connected to the joint. In some embodiments, the presence of one or more rigid strut joint can increase the resiliency of the frame structure 161. In some embodiments, a first strut 162 can be coupled non-rigidly to a second strut 162 at strut joint. For example, a strut joint can be a pin joint (e.g., wherein one or more strut 162 connected at the pin joint can rotate freely in a plane around the joint). In some embodiments, a non-rigid strut joint can improve the ability of frame structure 161 to assume an unexpanded configuration (e.g., inside of a delivery shaft or catheter sheath).

One or more strut 162 of the frame structure 161 can include a bend or angle. For example, one or more strut 162 of the frame structure 161 can include an arch, such as a distal arch or a proximal arch. In some embodiments, a bend or angle in a strut 162 (such as a proximal arch or a distal arch) can increase the resilience or structural strength of the frame structure 161. In some embodiments, the orientation of a bend or angle in a strut 162 can influence the directionality of the mechanical properties that it contributes to the frame structure 161. Proximal arches and distal arches that can provide circumferential resilience and or strength to the frame structure 161. In some embodiments, a bend or angle in a first strut 162 can extend a distance past a bend or angle in a second strut 162, longitudinally. In some embodiments, an arch can be an attachment point for a commissural strut.

The frame structure 161 can include one or more commissural strut. In some embodiments, the commissural strut can serve to join a first portion of the frame structure 161 with a second portion of the frame structure 161. In some embodiments, a commissural strut can connect to a first strut 162 and a second strut 162, wherein the first strut 162 defines at least a portion of a first expandable cell and second strut 162 defines at least a portion of a second expandable cell. In some embodiments, the commissural strut can be connected to a portion of a first section of expandable device 160 (e.g., leaflet hoop), for example, at a hoop joint. The commissural strut can be connected to a portion of frame structure 161 (e.g., a strut 162) and a portion of first section (e.g., a leaflet hoop). In some embodiments, the commissural strut can include a gap, slit, or hole (e.g., a commissural slit, a medial hole, or a distal hole). In some embodiments, the commissural strut can have a greater transverse cross-sectional area than a strut 162 of frame structure 161. In some embodiments, the commissural strut can be used to determine the rotational orientation of expandable device 160 around longitudinal axis 32, e.g., during insertion, placement, or anchoring of expanded valve device 160 at a target region. In some embodiments, the one or more commissural struts can be used to align the valve segment with the frame structure 161, for example, with each commissure of the plurality of valve leaflets being aligned with a respective commissural strut.

The frame structure 161 can include one or more expandable cells. For example, the frame structure 161 or portion thereof (e.g., a lattice structure of frame structure 161) can include 1, 2, 3, 4, 5, 6, 7, 8, 9, 10, from 10 to 20, from 20 to 30, from 30 to 40, from 40 to 50, from 50 to 100, from 100 to 150, from 150 to 200, or more than 200 expandable cells. The lattice structure or portion thereof can have a longitudinal length of from 1 mm to 50 mm, from 1 mm to 45 mm, from 1 mm to 40 mm, from 1 mm to 35 mm, from 1 mm to 30 mm, from 1 mm to 25 mm, from 1 mm to 20 mm, from 1 mm to 10 mm, from 10 mm to 45 mm, from 20 mm to 45 mm, from 20 mm to 30 mm, from 25 mm to 35 mm when prosthetic valve device 10 is in an unexpanded configuration. In some embodiments, a lattice structure or portion thereof can have a longitudinal length of from 1 mm to 45 mm, from 10 mm to 45 mm, from 15 mm to 45 mm, from 15 mm to 35 mm, from 16 mm to 34 mm, from 17 mm to 33 mm, from 18 mm to 32 mm, from 19 mm to 31 mm, from 20 mm to 30 mm, from 25 mm to 35 mm, or from 27.5 mm to 32.5 mm when expanded device 160 is in an expanded configuration. In some embodiments, an expandable cell of a lattice structure can be defined by a plurality of struts 162 (e.g., wherein the sides of the expandable cells are formed by struts 162). In some embodiment, an expandable cell can be defined by one or more strut and one or more additional structure, such as a specialized strut (e.g., a commissural post or leaflet hoop). In a representative example, an expandable cell of a frame structure 161 can be in plane with the struts 162 defining the expandable cell (e.g., oriented in a circumferential direction relative to the frame structure). An expandable cell of a frame structure 161 can be of any shape, including square, rectangular, circular, oval, trapezoidal, rhomboid, diamond-shaped, star-shaped, triangular, pentagonal, and hexagonal. The expandable cells of the frame structure 161 can be diamond-shaped, with the diamond-shaped expandable cells being circumferentially adjacent one another and the vertices of immediately adjacent expandable cells touching one another. An expandable cell of the frame structure 161 can have a first dimension. In some embodiments, an expandable cell of the frame structure 161 can be defined by a plurality of dimensions. For example, the expandable cell can have a first dimension and a second dimension. A representative example of a frame structure 161 includes a plurality of diamond-shaped expandable cells, the expandable cells being defined by a plurality of struts 162 coupled at strut joints and having first dimension and second dimension.

The frame structure 161 can include a single row of diamond-shaped expandable cells. However, it is contemplated that the frame structure 161 or a portion thereof (e.g., a lattice structure of frame structure 161) can include 1, 2, 3, 4, 5, 6, 7, 8, 9, 10, or more than 10 rows of expandable cells. In some embodiments, it can be advantageous to minimize the longitudinal length of a rigid portion of the frame structure 161 (e.g., for ease of introduction into a target region of a subject and/or for minimization of total amount of material required to form the frame structure). In some embodiments, the longitudinal length of the frame structure 161 or portion thereof can be minimized by minimizing the number of rows of expandable cells of frame structure 161 or a portion thereof (e.g., a lattice structure of the frame structure 161).

A method of using an expandable device 160 as described herein can include receiving a signal (e.g., a wireless signal) at the expandable device 160 (e.g., by the receiver wire 166). A signal received at the expandable device 160 can cause one or more actions. For example, a signal transmitted to expandable device 160 can cause one or more of the sensors 163 to make one or more measurements of one or more parameters (e.g., biological parameters).

A method of using of an expandable device 160 can include measuring (e.g., detecting) one or more physical, chemical, and/or biological parameter using the one or more sensors 163. For example, one or more sensors 163 of the expandable device 160 can be used to measure a physical, chemical, and/or biological parameter after being deployed at a target location of a subject. In some embodiments, one or more sensors 163 can be used to measure a strain (e.g., a deformation), a temperature, a fluid velocity, a force (e.g., a normal force or a shearing force), a pressure, a pH, or an oxygen concentration, e.g., at a target region of a subject.

In some embodiments, a sensor 163 of the expandable device 160 can be used to make or record a plurality of measurements over a period of time. For example, a sensor 163 can measure a variable that changes over time, such as a cyclical flow rate, cyclical strain value, or a strain rate. In some embodiments, a method of using the expandable device 160 can include making one or more measurements of one or more parameters (e.g., biological parameters) when a signal is received from an on-board circuit 165 or from an external device 171. In some embodiments, a method of using expandable device 160 can include making one or more measurements of one or more parameters (e.g., biological parameters) when the one or more parameters reaches or exceeds a threshold values.

A method of using an expandable device 160 disclosed herein can include storing data measured by the one or more sensors 163 in a transient or non-transient memory of one or more on-board circuits 165. In some embodiments, a signal received by the expandable device 160 (e.g., the receiver wire 166) can cause measured data (e.g., data measured by one or more sensors 163) to be stored in and/or transmitted from on-board circuit 165.

A method of using an expandable device 160 as disclosed herein can include transmitting stored data to an external receiver (e.g., via main wire 164). A signal from one or more sensors 163 (e.g., including data measured by the one or more sensors) can be processed by one or more on-board circuits 165 of expandable device 160. A frequency shift keying circuit can be used to send data (e.g., measured sensor data) to an external device 171 or portion thereof (e.g., external wire 170) via frequency-modulated transmitted signals. For example, an on-board circuit 165 can be used to convert a signal from one or more sensors 163 into a signal for transmission (e.g., to an external device 171) by encoding the data of the signal using frequency shift keying. In some cases, frequency shift keying can comprise modulating the frequency of a signal received by one or more wires (e.g., wires 164 or 166) of expandable device 160 to convey data of the one or more signals from a sensor 163 (or of data stored by on-board circuit 165) of the expandable device 160. Modulation of a frequency of a signal received by expandable device 160 (e.g., via one or more wires of expandable device 160) can be used to encode a binary signal in signal induced in one or more wires of expandable device 160 (e.g., by a wireless signal transmitted to expandable device 160 by one or more external wires 170). A frequency shift keying circuit can be configured to have a modulation frequency of from 1 kHz to 10 kHz, from 10 kHz to 20 kHz, from 20 kHz to 30 kHz, from 30 kHz to 40 kHz, or from 40 kHz to 50 kHz. For example, an expandable device 160 comprising a frequency shift keying circuit can be configured to respond to a 67 kHz signal received by transmitting signals of 67 kHz+/−10 kHz and 67 kHz+/−20 kHz for “on” and “off” signals. In some embodiments, frequency shift keying can be advantageous for transmission of a signal from an expandable device 160 to an external device 171 because the same wire (e.g., wires 164 or 166) of expandable device 160 can be used to receive a first signal (e.g., as encoded by on-off keying) and to transmit a second signal (e.g., sensor data encoded by frequency shift keying) with little or no interference to either signal or the need to deconvolve the signals.

In some embodiments, a signal can be encoded (e.g., by an on-off keying circuit of the expandable device 160) for transmission from an expandable device 160 to an external device 171 using on-off keying signal encoding. For example, an on-off keying circuit of the expandable device 160 can be used to send data (e.g., measured sensor data) to an external device or portion thereof (e.g., external wire 170) via on-off keying signals. In some embodiments, a signal encoded using frequency shift keying can be received by the expandable device 160 (e.g., from an external transmitter). In some embodiments, a frequency shift keying circuit can be used to convert a received signal (e.g., a signal encoded using frequency shift keying) to a digital signal.

A method of using an expandable device 160 can include receiving a signal (e.g., a signal transmitted by an external device). As mentioned above, a signal (e.g., a wireless signal) from an external device can be received by a component of expandable device 160. For example, one or more wires (e.g., wires 164 or 166) of the expandable device 160 can be used to receive a signal from an external device (e.g., through a current induction method such as transcutaneous energy transfer (TET)). A signal transmitted to the expandable device 160 can include instructions for the operation of one or more components of the expandable device 160, such as one or more sensors 163 and/or one or more on-board circuits 165. For example, a signal transmitted to the expandable device 160 can include triggering instructions for one or more sensors 163 and/or instructions for one or more on-board circuits 165. In some embodiments, instructions transmitted to the expandable device 160 can include instructions to make a measurement with a sensor 163 and/or to transmit the measured data from expandable device 160 (e.g., to an external device 171). Optionally, instructions transmitted to the expandable device 160 can include instructions to transmit stored data from the expandable device 160 (e.g., to an external device 171).

A signal transmitted to (e.g., received by) the expandable device 160 can be encoded using on-off keying. Encoding a signal using on-off keying can include modulating the amplitude of a signal with respect to time. In some embodiments, modulating the amplitude of a signal with respect to time (e.g., using on-off keying) can be used to transmit data in a binary signal (e.g., based on the length and number of signal sequences that are above or below a threshold amplitude). In some embodiments, a method disclosed herein can include converting an analog signal (e.g., as received by expandable device 160 from external device 171) to a digital signal. For example, a signal encoded by on-off keying that is received by one or more wires (e.g., wires 164, 166) of the expandable device 160 can be converted into a binary signal useful to a microcontroller of the on-board circuit 165 (e.g., by rectifying a sinusoidal signal and smoothing the signal using circuitry such as is shown in FIG. 4). In some embodiments, a signal received by the expandable device 160 can be a radio frequency (RF) signal. In some embodiments, a signal received by the expandable device 160 can be a signal induced in a wire (e.g., wire 164, 166) of the expandable device 160. In some embodiments, an analog signal received by the expandable device 160 can be used to power one or more components of the expandable device 160.

In some embodiments, one or more components of the expandable device 160 can be powered by a wireless signal (e.g., an analog wireless signal). For example, an electrical field or magnetic field can be used to directly or indirectly induce a current in a component (e.g., a wire or coil) of the expandable device 160. For example, inductive charging (e.g., transcutaneous energy transfer (TET)) or resonance charging can be used to induce a current in a component of the expandable device 160. In some embodiments, a method disclosed herein can include forming a current in an external device 171 or portion thereof (e.g., external wire 170). In some embodiments, a method disclosed herein can comprise positioning the external device 171 or portion thereof in close proximity to a subject in whom the expandable device 160 is deployed.

In some embodiments, a current can be induced in a wire of the expandable device 160 by forming a current in the external device 171 or portion thereof, e.g., as a result of a magnetic field formed by the current in the external and the orientation of the magnetic field relative to one or more wires (e.g., wires 164, 166) of the expandable device 160. For example, aligning an external wire 170 (e.g., a transmission coil) of an external device 171 with a wire (e.g., induction coil) of the expandable device 160 while a current is run through external wire 170 can induce a current in the aligned wire(s) of the expandable device 160. A current induced in one or more wires of the expandable device 160 can be used to power one or more components of the expandable device 160, such as one or more sensors 163 and/or one or more on-board circuits 165.

In some embodiments, increasing a magnetic field created by the external wire 170 can increase the current induced in the one or more wires (e.g., wires 164, 166). By increasing the current in the external wire 170, it can be possible to increase a magnetic field created by the external wire 170. In some embodiments, the strength of the magnetic field created by the external wire 170 (e.g., while a current is run through the external wire) can be increased if a magnetic core (e.g., a ferrite core) can be positioned in line with the field in the center of an induction circle or coil formed by the external wire 170. In some embodiments, increasing a magnetic field created by the external wire 170 can increase the distance that the external device 171 or portion thereof (e.g., external wire 170) can be positioned from the patient while still inducing a current in the one or more wires (e.g., wires 164, 166) of the expandable device.

To induce a current in one or more wires of the expandable device 160, an external device 171 or portion thereof (e.g., external wire 170) can be positioned adjacent to a subject (e.g., above, below, or to any side) or a portion of the subject in which the expandable device 160 is deployed. In some embodiments, an external device 171 or portion thereof can be positioned against a surface of the subject's body (e.g., skin or hair) during induction of a current in a wire (e.g., wires 164, 166) of the expandable device 160 and/or during transmission or receiving of a signal to or from the expandable device 160. In some embodiments, an external device 171 or portion thereof can be positioned against the fabric of the subject's clothing during induction of a current in a wire (e.g., wires 164, 166) of the expandable device 160 and/or during transmission or receiving of a signal to or from the expandable device 160. In some embodiments, an external device 171 or portion thereof (e.g., external wire 170) can be positioned at a distance of less than 1 cm, 1 cm to 5 cm, 5 cm to 10 cm, 10 cm to 20 cm, 20 cm to 50 cm, 50 cm to 100 cm, or more than 100 cm from a patient (e.g., to induce a current in one or more wires (e.g., wires 164, 166) of the expandable device 160 and/or to transmit or receive a signal to or from the expandable device 160).

The external wire 170 used to induce a current in one or more wires (e.g., wires 164, 166) of the expandable device 160 does not necessarily have to be the same external wire 170 that is used to transmit and/or receive a signal (e.g., a signal comprising instructions for an on-board circuit or sensor of expandable device 160) to or from expandable device 160. In some embodiments, a first external wire 170 used to induce a current in a wire of expandable device 160 can be remote from a second external wire 170 used to transmit and/or receive a signal to or from the expandable device 160. For example, a first external wire 170 (e.g., located in a hospital bed on which a subject is positioned) can be used to induce a current in one or more wires (e.g., wires 164, 166) of the expandable device 160, while a second external wire 170 (e.g., located in a hand-held wand in electrical connection with a system comprising the first external wire 170) can be used to transmit and/or receive a signal to or from the expandable device 160. In some embodiments, a first external wire 170 (e.g., used for inducing a current in one or more wires of expandable device 160) can include a portion of an external device 171 that is separate from an external device 171 that comprises second external wire 170 (e.g., used for transmission and/or receiving of one or more signals from expandable device 160). Configurations in which a first external wire 170 (e.g., used for inducing a current in one or more wires of the expandable device 160) and a second external wire 170 (e.g., used for transmission and/or receiving of one or more signals from expandable device 160) are remote from one another or wherein the first and second external wires comprise portions of separate external devices can improve the versatility of a system described herein, for example, by allowing measurements to be taken continuously while data is transferred to an external device (e.g., for analysis) at discrete time points (e.g., by a technician or nurse).

A signal transmitted using the expandable device 160 (e.g., via wires 164, 166) can be received by external wire 170. For example, a signal comprising sensor data transmitted using the expandable device 160 or a portion thereof can be received by an external wire 170 of external device 171. A signal received transmitted by the expandable device 160 and received by the external wire 170 can be an analog or a digital signal. In some embodiments, a method disclosed herein can include receiving a signal transmitted using an expandable device 160 that is encoded (e.g., using a technique disclosed herein, such as frequency shift keying). A signal received by the external wire 170, a signal (e.g., an analog encoded signal) can be converted from an analog signal to a digital signal. For example, a processor of external device 171 (which can, optionally, be directly coupled to external wire 170) can be used to convert an analog signal to a digital signal. A method disclosed herein can include displaying a digital signal (e.g., a signal transmitted by expandable device 160, which has, optionally, been converted into a digital signal after having been received by external wire 170) on a visual display. In some embodiments, a signal (e.g., a signal comprising sensor data) can be stored using a non-transient memory of external device 171. In some embodiments, a signal transmitted by the expandable device 160 can be a radio frequency (RF) signal. In some embodiments, a signal transmitted by the expandable device 160 can be a signal induced in a wire of expandable device 160.

Any of the expandable devices herein (e.g., expandable device 160), including any of the frame structures herein (e.g., frame structure 161), can be used as part of a heart valve prosthesis. The expandable devices 160 described herein can be used to treat a heart of a subject (e.g., a patient). In some embodiments, treating a subject can include repairing or replacing a valve of the subject, such as a heart valve (e.g., a mitral valve, a pulmonary valve, a tricuspid valve, or an aortic valve). The expandable device 160 can be introduced into a subject (e.g., via a delivery device such as a catheter, which may comprise an outer sheath). In some embodiments, the expandable device 160 can be delivered transseptally or transapically. In some embodiments, treating a subject (e.g., patient) can comprise positioning the expandable device 160 in a native valve of the subject. Structures and devices described herein (e.g., self-expandable, malecot-expandable, or balloon-expandable prosthetic valve devices and frame structures; anchors; barbs; hooks; coil grabbers) can be used to secure the expandable device 160 in a target region or location, such as within a native valve of a subject.

The distal end of a delivery device (e.g., for the expandable device 160) may be configured to be advanced from a first side of a native valve to a second side of the native valve. For example, the distal end of the delivery device may be advanced from a left atrial side of a mitral valve to a left ventricular side of a mitral valve. In some instances, the distal end of the delivery device may be transseptally inserted into the left atrium of the heart prior to advancement into the left ventricle. Alternatively, or in combination, the distal end of the delivery device may be steerable such that it is positionable to point towards the first side of the native valve before being advanced to the second side of the native valve. A steerable delivery device can be particularly useful in cases when the delivery device is navigated through a tortuous path during deployment of an expandable device disclosed herein. The placement and/or orientation of components (e.g., one or more sensors, on-board circuits, or wires, such as a transmission coil or an antenna) can decrease the likelihood that a portion of the expandable device will be damaged or thrown out of alignment in the process of directing the expandable device to a target location (e.g., via a tortuous path through the body).

In some embodiments, the expandable device 160 may further include an anchor, such as a spiral anchor. Fully deploying an anchor of the expandable device 160 may comprise actuating the anchor from an elongated delivery configuration to a deployed (e.g., spiral) configuration on the first side of the native valve and advancing the anchor in the deployed configuration through the native valve to the second side of the native valve. Advancing the anchor may comprise pushing the anchor through the native valve. Advancing the anchor may further comprise rotating the anchor through the native valve. In some embodiments, fully deploying the anchor may include positioning the anchor such that it is located only on the second side of the native valve. In some embodiments, the anchor may be actuated from the delivery configuration to the deployed configuration on a first side of the native valve prior to being advanced to a second side of the native valve. For example, the anchor may be deployed in a left atrium of a heart prior to being advanced to a left ventricle of the heart as described herein. In some embodiments, the anchor may be actuated from the delivery configuration to the deployed configuration on a second side of the native valve after being advanced to the second side from a first side of the native valve. For example, an anchor may be advanced from a left atrium of a heart prior to being deployed in a left ventricle of the heart. In some embodiments, the anchor can be deployed after the rest of the expandable device 160 (e.g., the frame structure 161) has been deployed in a target region of the subject. For example, an anchor can be advanced through an end (e.g., a distal end) of the frame structure 161 when the frame structure 161 is in an expanded configuration. Advancing the anchor can include pushing the anchor through the native valve and/or rotating the anchor. An anchor can be configured (e.g., through its spiral shape) to wrap at least partially around expandable device 160 or a portion thereof. For example, the anchor can be configured to wrap at least partially around the frame structure 161.

A free end of a deployed anchor may optionally be rotated around one or more structures on the second side of the native valve. The one or more structures may comprise one or more valve leaflets of the native valve. Alternatively, or in combination, the one or more structures may comprise one or more chordae of the left ventricle. The free end of the deployed anchor may optionally rotated around one or more structures on the second side of the native valve such that the one or more structures (e.g., chordae, leaflets, or annulus) are pulled radially inwards towards the longitudinal axis of the anchor and/or towards the longitudinal axis of the delivery device. The anchor or a portion thereof (e.g., free end of the anchor) may be configured such that minimal torque is applied to the one or more structures, for example, when the anchor or portion thereof engages with the one or more structures. Alternatively, or in combination, the anchor and/or free end may be configured such that the one or more structures are not rotated, or are minimally rotated, during rotation of the anchor. An anchor can comprise a proximal end opposite free end, which may be used to couple the anchor to expandable device 160 or a portion thereof. In some embodiments, the anchor can then be released from the distal end of the delivery device. The anchor may be released from the distal end of the delivery device on the second side of the native valve.

In some embodiments, the expandable device 160 or a portion thereof (e.g., frame structure 161) may be released from the distal end of the same delivery device used to delivery the anchor or from the distal end of a separate delivery device. In some embodiments, at least a portion of the frame structure 161 may be expanded within at least a portion of the deployed anchor to anchor the frame structure to the native valve. In some embodiments, expanding the frame structure 161 and releasing the frame structure 161 may occur simultaneously. In some embodiments, the delivery device can be retracted from the native valve after the expandable device 161 or portion thereof is released from the delivery device.

The delivery device may include an inner shaft as described herein. The delivery device may optionally comprise an outer shaft, a guidewire, and/or an inflatable balloon, in any combination thereof as desired by one of ordinary skill in the art. A distal end of the delivery device may be inserted into the left atrium of the heart via a transseptal puncture as described herein. For example, the distal ends of inner shaft and/or outer sheath may be advanced into the left atrium of the heart. The inner shaft may optionally be advanced distally into the left atrium away from the distal end of the outer sheath. In some embodiments, advancing the inner shaft relative to the outer sheath may aid in deployment and/or placement of the expandable device 160 as described herein. In some embodiments, both the inner shaft and the outer sheath may be advanced distally into the left atrium through the transseptal puncture.

At least a portion of the expandable device 160 may be deployed from an unexpanded (for example, compressed or undeployed) configuration to an expanded configuration within the left atrium. At least a portion of the anchor may be deployed from a delivery and/or elongated configuration to a deployed configuration within the heart. For example, the anchor may be actuated from an elongated configuration to a deployed configuration within the left atrium. In some embodiments, the anchor may be deployed from the inner shaft by pushing the anchor out of the inner shaft, releasing the anchor from radial constraint by retracting the outer sheath or the like, as described herein. After the anchor has been deployed from the delivery device, the frame structure 161 may be at least partially deployed from the delivery device so as to place the frame structure 161 within the anchor. The frame structure 161 may be deployed from the delivery device in either the unexpanded configuration or the expanded configuration, depending on the location of deployment, as will be understood by one of ordinary skill in the art based on the teachings herein.

The distal end of the delivery device (for example, the distal end of the inner shaft and/or the outer sheath) may be steered such that the distal end of the delivery device points toward the atrial side of the native valve. Such steering may occur prior to, during, or after deployment of at least a portion (for example deployment of an anchor) of the expandable device 160. In some embodiments, the distal end of the outer sheath may be steerable. Alternatively, or in combination, the inner shaft may include a joint configured to change an angle of the distal portion of the inner shaft relative to a proximal portion of the inner shaft. In some embodiments, the inner shaft can be steered by changing the angle of the distal portion of the inner shaft relative to the proximal portion of the inner shaft. The angle of the joint may be changed passively or actively. In various embodiments, the angle may be selectively controlled by a proximal handle. For example, pull wires or other mechanisms may connect to controls on the handle and to the joint or the distal end of the delivery device, e.g., for controlling an angle of the joint.

The expandable device 160 may be advanced through the native valve by the delivery device from the left atrium to the left ventricle. Advancement of the expandable device 160 and optionally delivery device through the mitral valve may be facilitated by the natural opening and closing of the valve during the cardiac cycle. The distal end of the delivery device and/or expandable device 160 may be configured to be advanced from a first side of a native valve to a second side of the native valve. For example, the distal end of the delivery device and/or the expandable device 160 may be advanced from a left atrial side of a mitral valve to a left ventricular side of a mitral valve. Advancing the anchor may comprise pushing the anchor through the native valve. Alternatively, or in combination, advancing the anchor may include rotating the anchor through the native valve. In some embodiments, the combination of rotational motion and pushing may facilitate advancement of the device from the first side of the native valve to the second side of the native valve. Rotation of the expandable device 160, for example rotation of the anchor and/or frame structure 161, may be facilitated by the inner shaft. For example, the inner shaft may transmit rotational motion to the expandable device 160 in order to rotate the expandable device 160 during advancement through the native valve.

Rotation of the anchor during advancement may assist with a process of stretching the anchor into an extended or expanded configuration by aiding in unwinding the anchor. Additionally, the rotational motion may reduce the risk of the free end of the anchor unintentionally engaging other structures of the subject's anatomy during insertion through the native valve leaflets. The anchor may be sufficiently elastic so as to enable relatively easy insertion through the native valve and/or reduce the risk of injury to the native leaflets. After the anchor has stretched through the native valve, it may return to the deployed configuration.

In some embodiments, the anchor may be advanced to the ventricle before being deployed from the delivery (e.g., elongated) configuration to the deployed configuration.

One or more structures on the ventricular side of the native valve may include one or more valve leaflets and/or one or more chordae tendineae. After the anchor has been at least partially deployed within the left ventricle adjacent one or more chordae tendineae, the expandable device 160 and/or a portion thereof (e.g., the anchor) may be rotated to capture and anchor the native chordae and/or native leaflets. The free end of the anchor may extend radially outward from the rest of the anchor to facilitate capture of the native structures. The free end of the anchor may be rotated around one or more of the chordae tendineae. Additional rotation of the anchor may gradually capture additional chordae tendineae.

Rotation of the expandable device 160, for example, rotation of the anchor and/or frame structure 161, may be facilitated by the delivery device. For example, the inner shaft may be rotated and rotational motion may be transmitted from the inner shaft to the expandable device 160 in order to rotate the expandable device 160 around one or more of the structures on the ventricle side of the mitral valve as described herein.

A portion of expandable device 160 (e.g., the anchor) may be wrapped around the captured chordae tendineae. The expandable device 160 may be rotated around the chordae tendineae such that the chordae tendineae are pulled inwardly into bunches (e.g., by an anchor structure of expandable device 160). The native valve leaflets may also be in communication with the expandable device 160. The expandable device 160 (e.g., the anchor) may be rotated to capture enough chordae tendineae and/or valve leaflets to rigidly anchor the anchor adjacent the native valve annulus. The expandable device 160 or a portion thereof (e.g., the anchor) may be anchored by wrapping around only a portion of the chordae. Although it may be possible to capture all or substantially all the chordae, this may not be necessary to provide sufficient anchoring of the expandable device 160. The expandable device 160 may be further anchored by expansion of the frame structure 161 within the native valve and against the anchor.

In some embodiments, in addition to or as an alternative to the spiral anchor described herein, the expandable device 160 can be anchored to a structure of the native tissue environment (e.g., a native valve or portion thereof, one or more chordae tendinae, or wall of a heart chamber) by engaging one or more hooks, barbs, and/or scallop-shaped anchors coupled to the expandable device 160 with the structure of the native tissue environment (e.g., a portion of a native valve). For example, the expandable device 160 may be secured at a target region of a subject by engaging one or more barbs of the expandable device 160 with a structure of the native tissue environment.

In some embodiments, once the spiral anchor has been anchored adjacent to the native valve, the frame structure 161 and a valve segment of expandable device 160, which can comprise valve leaflets directly or indirectly coupled to frame structure 161) may be expanded at least partially within the anchor as described herein. The frame structure 161 and the valve segment may be deployed (e.g., expanded) simultaneously. Alternatively, or in combination, the frame structure 161 and the valve segment may be deployed sequentially, for example by first expanding the frame structure 161 and then receiving the valve segment therein.

The frame structure 161 may be expanded within the native valve from an unexpanded configuration to an expanded configuration. In some embodiments, at least a portion the frame structure 161 may be expanded within at least a portion of the deployed an anchor to anchor the frame structure 161 to the native valve. In some embodiments, the frame structure 161 may comprise an expandable stent comprising one or more sensors, one or more on-board circuits, and/or one or more wires (e.g., antennas and/or receiving coils). In some embodiments, the frame structure 161 of expandable device 160 may be self-expandable (e.g., self-expanding). In some embodiments, the frame structure 161 of expandable device 160 may be balloon-expandable. The delivery device may include a balloon that may be disposed within the expandable device 160 in order to expand the expandable device 160. The balloon may be positioned within at least a portion of the expandable device 160, for example within at least a portion of frame structure 161 in an uninflated configuration prior to being inflated. The inflatable balloon may, for example, be disposed within the inner shaft or outer sheath of the delivery device while the anchor is being positioned adjacent the native valve and then advanced therefrom (or the inner shaft or outer sheath is retracted therefrom) to be positioned within the frame structure 161. In some embodiments, the inflatable balloon may be disposed within the frame structure 161 during placement of the expandable device 160. Frame structure 161 may be partially expanded towards the anchor in order to capture the chordae tendineae therebetween. As the frame structure 161 continues to be expanded to a fully expanded state the chordae tendineae may be sandwiched between the anchor and the frame structure 161. The frame structure 161 and anchor may thus be anchored to the chordae tendineae.

The expandable device 160 can be released from the delivery device, e.g., after anchoring. In some embodiments, releasing the expandable device 160 may comprise releasing the anchor and/or the frame structure 161. Releasing the expandable device 160 from the delivery device may include expanding the expandable device 160 from the unexpanded configuration to the expanded configuration. For example, expanding the frame structure 161 and releasing the frame structure 161 may occur simultaneously as described herein. Alternatively, the frame structure 161 may be released prior to or after being expanded.

A method of using an expandable device 160, as disclosed herein, may comprise deflation of the balloon, retraction of the balloon into inner shaft, and/or removal of the delivery device from the heart. After the frame structure 161 has been expanded and anchored to the native valve as described herein, the inflatable balloon may be deflated. The balloon may optionally be retracted back into the delivery device, for example into inner shaft. The delivery device may then be removed from the heart.

Although the steps above describe a method of deploying an expandable device 160 within a native valve in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as necessary to assemble at least a part of an article.

For example, in some embodiments, deploying the expandable device 160 may occur in multiple steps such that a portion of the expandable device 160 (e.g., an anchor) may be deployed before another portion the expandable device 160 (e.g., frame structure 161). Alternatively, or in combination, in some embodiments, deploying the anchor may occur in multiple steps such that a portion of the anchor may be deployed before being advanced through the native valve and another portion of the anchor may be deployed after being advanced through the native valve. Alternatively, or in combination, the delivery device may be advanced from the left atrium to the left ventricle with the expandable device 160 undeployed (e.g., in an unexpanded configuration). In many embodiments, the frame structure may be self-expanding and the balloon may not be necessary for expansion of the frame structure 161. Alternatively, or in combination, the anchor may be released after the frame structure 161 has been expanded within it.

Any of the expandable devices herein (e.g., expandable device 160), including any of the frame structures herein (e.g., frame structure 161), may be a part of an expandable blood pump housing, or shroud, that at least partially surrounds one or more rotatable impellers that when rotated causes blood to move through the expandable housing/shroud. For example, any of the expandable devices herein that include one more of at least one sensor (e.g., sensors 163), a main wire (e.g., main wire 164), on-board circuits (e.g., circuits 165), or receiver wires (e.g., receiver wire 166) may be a part of an expandable blood pump housing, or shroud, that at least partially surrounds one or more rotatable impellers that when rotated causes blood to move through the expandable housing/shroud. For example, any of the expandable devices herein can be part of an impeller assembly, such as impeller assembly 92 described in U.S. Pat. No. 10,029,037, which is incorporated by reference herein for all purposes. For example, any of the sensors, main wires, on-board circuits, and/or receiver wires herein can be mounted to any of the cannulas in U.S. Pat. No. 10,029,037, such as being mounted to one or more circumferential rings 55. That is, circumferential rings in U.S. Pat. No. 10,029,037 may be considered part of or define a frame structure, as that phrase in used herein. Any of the sensors, main wires, on-board circuits, and/or receiver wires herein may be coupled to one or more connectors (such as connector 541 in U.S. Pat. No. 10,029,037) that extend proximally therefrom, and may extend to a proximal location that remains outside of a patient when in use, such as to an external console or other similar control-type device/system. The external console may receive information conducted through the one or connectors, such as information sensed by a sensor that is mounted to the expandable device frame structure. Any and all uses of information that is sensed by any sensor described in U.S. Pat. No. 10,029,037 is explicitly incorporated by reference herein for all purposes including using any of the described expandable devices.

As set forth herein, any of the expandable devices herein may be a part of pump portion of a blood pump, such as part of an expandable impeller housing/shroud (also referred to in some cases as a cannula). The pump portion can be advanced intravascularly (e.g., with femoral artery access) to a target location in a patient, such as adjacent an aortic valve. Any and all methods of delivery and placement of a pump portion of an intravascular blood pump described in U.S. Pat. No. 10,029,037 are expressly included herein by reference for all purposes. After the pump portion has been advanced to a target location, one or more connectors may extend proximally from a sensor and to a location outside of the patient, where it may be coupled to an external device so that it may communicated information (e.g., sensed information, such as information indicative of blood pressure) to the external device.

Various expandable devices or components thereof, sensors, sensor circuitry, and/or other elements are described in the following, all of which are incorporated by reference in their entireties: U.S. Pat. Nos. 5,873,835, 6,053,873, 9,343,224, U.S. Pat. Nos. 9,474,840, 10,029,037, and U.S. Patent Publication No. 2017/0258585.

It should be understood that any feature described herein with respect to one embodiment can be used in addition to or in place of any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An expandable device comprising:
a frame structure having an expanded configuration and an unexpanded configuration;
one or more sensors directly coupled to one or more struts of the frame structure; and
one or more antennas coupled to the one or more sensors.

2. The expandable device of claim 1, wherein the one or more antennas are coupled directly to the frame structure.

3. The expandable device of claim 1, wherein at least one surface of an antenna of the one or more antennas comprises a non-conducting coating.

4. The expandable device of claim 1, wherein an antenna of the one or more antennas comprises an induction coil.

5. The expandable device of claim 1, further comprising one or more on-board circuits.

6. The expandable device of claim 5, wherein an on-board circuit of the one or more on-board circuits is electrically coupled to the one or more sensors.

7. The expandable device of claim 5, wherein the one or more on-board circuits comprise a processor configured to convert data received from the one or more sensors into one or more transmission signals.

8. The expandable device of claim 1, wherein the one or more on-board circuits comprise a processor configured to operate the one or more sensors based on a signal received by the one or more antennas.

9. The expandable device of claim 1, wherein the frame structure comprises a plurality of struts coupled at a plurality of strut joints.

10. The expandable device of claim 9, wherein each sensor of the one or more sensors is coupled to the frame structure between two strut joints.

11. The expandable device of claim 9, wherein each on-board circuit of the one or more on-board circuits is coupled to the frame structure between two strut joints.

12. The expandable device of claim 10, wherein the two strut joints are adjacent strut joints.

13. The expandable device of claim 1, comprising a plurality of sensors.

14. The expandable device of claim 13, wherein a first sensor of the plurality of sensors is coupled to the frame structure closer to a first longitudinal end of the frame structure than to a second longitudinal end of the frame structure, and a second sensor of the plurality of sensors is coupled to the frame structure closer to the second longitudinal end of the frame structure than to the first longitudinal end of the frame structure.

15-27. (canceled)

28. A method of deploying an expandable device, the method comprising:
delivering an expandable device to a target region of a subject, the expandable device comprising a frame structure, one or more sensors coupled directly to the frame structure, and one or more antennas coupled directly to the frame structure; and
expanding the expandable device at the target region of a subject.

29. The method of claim 28, comprising:
receiving an encoded analog signal using an antenna of the expandable device;
converting the signal from an analog signal to a digital signal; and
operating one or more sensors coupled directly to the expandable device based on the digital signal.

30. The method of claim 29, further comprising transmitting the analog signal to the expandable device using an external device.

31. The method of claim 30, further comprising encoding the analog signal prior to transmitting the analog signal to the expandable device.

32. The method of claim 30, wherein the analog signal is transmitted wirelessly.

33. The method of claim 29, wherein the analog signal is received wirelessly.

34-53. (canceled)
